SEQUENCE LISTING (10 sequences)

SEQ ID NO: 1 - DNA, 1275 bp, Bacteroides fragilis
gcgcgattactagcgaatccagcttcacgaagtcgggttgcagacttcgatccgaactgagagaggattttgggattagcatacggtcacccgctagctgccttctgtaccccccattgtaacacgtgtgtagccccggacgtaagggccgtgctgatttgacgtcatccccaccttcctcacatcttacgacggcagtctctccagagtcctcagcatgacctgttagtaactgaagataagggttgcgctcgttatggcacttaagccgacacctcacggcacgagctgacgacaaccatgcagcaccttcacagcggtgattgctcactgacatgtttccacatcattccactgcaatttaagcccgggtaaggttcctcgcgtatcatcgaattaaaccacatgttcctccgcttgtgcgggcccccgtcaattcctttgagtttcaccgttgccggcgtactccccaggtggaatacttaatgctttcgcttggccgcttactgtatatcgcaaacagcgagtattcatcgtttactgtgtggactaccagggtatctaatcctgtttgatacccacactttcgagcatcagtgtcagttgcagtccagtgagctgccttcgcaatcggagttcttcgtgatatctaagcatttcaccgctacaccacgaattccgcccacctctactgtactcaagactgacagtatcaactgcaattttacggttgagccgcaractttcacaactgacttaccagtccacctacgctccctttaaacccaataaatccggataacgctcggatcctccgtattaccgcggctgctggcacggagttagccgatccttattcatataatacatacaaaacagtatacatactgcactttattcttatataaaagaagtttacgacccatagagccttcatccttcacgctacttggctggttcaggctagcgcccattgaccaatattcctcactgctgcctcccgtaggagtttggaccgtgtctcagttccaatgtgggggaccttcctctcagaacccctatccatcgaaggcttggtgagccgttacctcaccaacaacctaatggaacgcatccccatcctttaccggaatcctttaataatgaaaccatgcggaatcattatgctatcgggtattaatctttctttcgaaaggctatccccgagtaaagggcaggttggatacgtgttactcacccgtgcgccggtcgccggcaaagaaagcaagctttctt

SEQ ID NO: 2 - protein, 405 aa, Bacteroides fragilis
MFILNFNKMKNVKLLLMLGTAALLAACSNEADSLTTSIDAPVTASIDLQSVSYTDLATQLNDVSDFGKMIILKDNGFNRQVHVSMDKRTKIQLDNENVRLFNGRDKDSTSFILGDEFAVLRFYRNGESISYIAYKEAQMMNEIAEFYAAPFKKTRAINEKEAFECIYDSRTRSAGKDIVSVKINIDKAKKILNLPECDYINDYIKTPQVPHGITESQTRAVPSEPKTVYVICLRENGSTIYPNEVSAQMQDAANSVYAVHGLKRYVNFHFVLYTTEYSCPSGDAKEGLEGFTASLKSNPKAEGYDDQIYFLIRWGTWDNKILGMSWFNSYNVNTASDFEASGMSTTQLMYPGVMAHELGHILGAEHTDNSKDLMYATFTGYLSHLSEKNMDIIAKNLGWEAADGD

SEQ ID NO: 3 - protein, 397 aa, Bacteroides fragilis
MKNVKLLLMLGTAALLAACSNEADSLTTSIDTPVTASIDLQSVSYTDLATQLNDVSDFGKMIILKDNGFNRQVHVSMDKRTKIQLDNENVRLFNGRDKDSTSFILGDEFAVLRFYRNGESISYIAYKEAQMMNEIAEFYAAPFKKTRAINEKEAFECIYDSRTRSAGKDLVSVKINIDKAKKILNLPECDYINDYIKTPQVPHGITESQTRAVPSEPKTVYVICLRESGSTVYPNEVSAQMQDAANSVYAVHGLKRFVNLHFVLYTTEYSCPSGNADEGLDGFTASLKANPKAEGYDDQIYFLIRWGTWDNNILGISWLDSYNVNTASDFKASGMSTTQLMYPGVMAHELGHILGARHADDPKDLMYSKYTGYLFHLSEENMYRIAKNLGWEIADGD

SEQ ID NO: 4 - protein, 397 aa, Bacteroides fragilis
MKNVKLLLMLGTAALLAACSNEADSLTTSIDAPVTASIDLQSVSYTDLATQLNDVSDFGKMIILKDNGFNRQVHVSMDKRTKIQLDNENVRLFNGRDKDSTNFILGDEFAVLRFYRNGESISYIAYKEAQMMNEIAEFYAAPFKKTRAINEKEAFECIYDSRTRSAGKYPVSVKINVDKAKKILNLPECDYINDYIKTPQVPHGITESQTRAVPSEPKTVYVICLRENGSTVYPNEVSAQMQDAANSVYAVHGLKRYVNLHFVLYTTEYACPSGNADEGLDGFTASLKANPKAEGYDDQIYFLIRWGTWDNNILGISWLNSYNVNTASDFKASGMSTTQLMYPGVMAHELGHILGANHADDPKDLMYSKYTGYLFHLSEKNMDIIAKNLGWEIADGD

SEQ ID NO: 5 - DNA, 20 bp, Artificial Sequence
  PCR primer 368-F
gaacctaaaacggtatatgt

SEQ ID NO: 6 - DNA, 20 bp, Artificial Sequence
  PCR primer 368-R
gttgtagacatcccactggc

SEQ ID NO: 7 - DNA, 22 bp, Artificial Sequence
  PCR primer Bft-F
ggatacatcagctgggttgtag

SEQ ID NO: 8 - DNA, 20 bp, Artificial Sequence
  PCR primer Bft-R
gcgaactcggtttatgcagt

SEQ ID NO: 9 - DNA, 29 bp, Artificial Sequence
  PCR primer BFTF
cgcggcattattagctgcatgttctaatg

SEQ ID NO: 10 - DNA, 29 bp, Artificial Sequence
  PCR primer P4
gatacatcagctgggttgtagacatccca

(19) United States
(12) Patent Application Publication
(10) Pub. No.: US 2021/0261651 A1
(43) Pub. Date: Aug. 26, 2021

(54) COMPOSITIONS AND METHODS FOR TREATING INFLAMMATORY BOWEL DISEASE

(71) Applicant: ARTIZAN BIOSCIENCES, New Haven, CT (US)

(72) Inventor: Whitman SCHOFIELD, New Haven, CT (US)

(21) Appl. No.: 17/253,333

(22) PCT Filed: Jul. 3, 2019
(86) PCT No.: PCT/US2019/040601

§ 371 Date: Dec. 17, 2020

Related U.S. Application Data
(60) Provisional application No. 62/693,798, filed on Jul. 3, 2018.

(51) Int. Cl. C07K 16/12; A61K 39/02; A61K 39/39; A61K 9/00; A61P 1/00; C12Q 1/689

(52) U.S. Cl. CPC C07K 16/1257; A61K 39/0216; A61K 39/39; A61K 9/0053; A61K 2039/505; A61P 1/00; C12Q 1/689; C07K 2317/24; C07K 2317/76; A61K 9/0019

(57) ABSTRACT

Described are compositions and methods for treating inflammatory bowel diseases in a subject in need thereof. In certain aspects, the disclosure provides methods of treating a subject diagnosed with Irritable Bowel Disease (IBD), the method comprising administering to the subject an agent to reduce the number or pathogenic effects of a B. fragilis strain, wherein the subject is diagnosed with IBD by detecting the presence of the B. fragilis strain or a B. fragilis toxin in a biological sample of the patient.

### CROSS-REFERENCE TO RELATED APPLICATIONS

This application claims priority to U.S. Provisional Application Serial No. 62/693,798, filed Jul. 3, 2018, which is incorporated by reference herein in its entirety for all purposes.

### SEQUENCE LISTING

This application contains a Sequence Listing which has been submitted electronically and is hereby incorporated by reference in its entirety. The Sequence Listing was created Jul. 2, 2019, is named ARTI_003_01_WO_SeqList_ST25.txt and is ˜13.3 Kilobytes in size.

### FIELD OF THE DISCLOSURE

The present disclosure relates to compositions and methods for preventing or treating inflammatory bowel disease. More particularly, the present disclosure relates to compositions and methods for reducing the number or pathogenic effects of at least one type of bacteria associated with inflammatory bowel disease.

### BACKGROUND

Inflammatory bowel disease (IBD) is a heterogeneous disease that includes ulcerative colitis (UC) and Crohn's disease (CD). Symptoms of IBD may include diarrhea, cramping, abdominal pains, weight loss, rectal bleeding, tiredness, anemia, fistulae, perforations, obstruction of the bowel and frequently, the need for surgical intervention. According to the US Centers for Disease Control and Prevention, about 1.4 million people in the US suffer from IBD, making it one of the most prevalent gastrointestinal diseases in the country. The overall healthcare cost of IBD in the US is estimated to be more than $1.7 billion per year.

IBD has no cure. Current therapies for IBD are directed at reducing the inflammatory process and at reducing the detrimental effects of the inflammatory process associated with the disease. Therapies may include administration of anti-inflammatory drugs (e.g., mesalamine, sulfasalazine, infliximab, adalimumab, prednisone, and/or budesonide) and immunosuppressive drugs (e.g., 6-mercaptopurine, azathioprine, and/or cyclosporine). Such therapies are often associated with adverse side effects, such as nausea, vomiting, anorexia, dyspepsia, malaise, headaches, abdominal pain, fever, rash, pancreatitis, bone marrow suppression, formation of antibodies, infusion reactions, and increased opportunistic infections.

Accordingly, there is a need in the art for improved treatments for IBD.

### SUMMARY

The disclosure relates to compositions and methods for preventing or treating inflammatory bowel disease (IBD) in a subject in need thereof.

In some aspects, the disclosure relates to methods of treating a subject diagnosed with IBD, the methods comprising administering to the subject an agent to reduce the number or pathogenic effects of a B. fragilis strain, wherein the subject is diagnosed with IBD by detecting the presence of the B. fragilis strain in a biological sample of the patient. The agent may be, for example, an antibody or a vaccine.

In some aspects, the disclosure relates to methods of treating a subject diagnosed with IBD, the methods comprising administering to the subject an agent to reduce the expression or activity of a B. fragilis toxin, wherein the subject is diagnosed with IBD by detecting the presence of the B. fragilis toxin in a biological sample of the patient.

In some aspects, the disclosure relates to a method of treating a subject in need thereof, the method comprising detecting the presence of a B. fragilis strain in a biological sample of the subject, and administering to the subject an agent to reduce the number of or pathogenic effects of the B. fragilis strain.

In some aspects, the disclosure relates to a method of treating a subject in need thereof, the method comprising detecting the presence of the B. fragilis toxin in a biological sample of the subject, and administering to the subject an agent to reduce the expression or activity of the B. fragilis toxin.

In some aspects, the disclosure relates to immunogenic compositions (e.g., vaccines) to reduce the number or pathogenic effects of one or more bacteria associated with the development or progression of an inflammatory bowel disease.

In some aspects, the disclosure provides a method of treating or preventing an inflammatory bowel disease in a subject, the method comprising administering to the subject an agent to reduce the number or pathogenic effects of a B. fragilis strain.

In some aspects, the disclosure provides a method of treating or preventing an inflammatory bowel disease in a subject, the method comprising administering to the subject an agent to reduce the number or pathogenic effects of an enterotoxigenic B. fragilis strain.

In some aspects, the disclosure provides a vaccine composition for treating or preventing an inflammatory bowel disease, the vaccine composition comprising an inactivated B. fragilis enterotoxin.

In some aspects, the disclosure provides a method of treating or preventing an inflammatory bowel disease in a subject in need thereof, the method comprising administering to the subject an agent to reduce the effects of an enterotoxin produced by a B. fragilis strain.

These and other aspects are described in greater detail below.

### BRIEF DESCRIPTION OF THE DRAWINGS

FIG. 1. FIG. 1 is a graph showing that ETBF strains isolated from IBD patients cause cecal inflammation upon monocolonization. The graph depicts cecal weight of mice colonized with either an NTBF type strain (ATCC® 25285), or an ETBF strain isolated from an IBD patient (# 1, # 2, or # 3). Statistically significant differences between NTBF and ETBF strains are indicated with asterisks (one-way ANOVA followed by Dunnett's multiple comparisons test, **** =adjusted p value <0.0001, N=3-4).

FIG. 2. FIG. 2 shows histology score of cecal tissue from gnotobiotic mice colonized with a NTBF-type strain (ATCC® 25285), an ETBF-type strain (ATCC® 43859), and ETBF strains (# 1, # 2, or # 3) isolated from IBD patients. The histology scores were assigned based on degree of inflammation (mild, moderate, severe), extent of epithelial damage (focal erosions, erosions, extended ulcerations/granulation tissue/pseudopolyps), and percent area involved (10-25%, 25-50%, 50-75%, and 75-100%). Scores increase with severity. Asterisks indicate statistical significance, as determined using One-way ANOVA followed by Dunnet's multiple comparison test (* =p<0.05, ** =p <0.005, **** =p <0.0001).

FIG. 3. FIG. 3 provides representative data showing fecal lipocalin2 levels in mice colonized with a NTBF strain or an ETBF strain isolated from an IBD patient in various microbiota background settings. Asterisks indicate statistical significance, as determined using the Two-way ANOVA Sidak's multiple comparison test (* =p <0.05, ** =p<0.01).

FIG. 4A-4B. FIG. 4A-4B show levels of pro-inflammatory cytokines IL-17 (FIG. 4A) and TNF (FIG. 4B) in cecal tissue explants from gnotobiotic mice colonized with NTBF or ETBF strains isolated from IBD patients. Asterisks indicate statistical significance, as determined using t-test (* =p<0.05, ** =p<0.01).

FIG. 5A-5B. Provided are representative histological images of cecal tissues of mice colonized with NTBF (FIG. 5A) or ETBF (IBD isolate, FIG. 5B) in a synthetic microbiota background. Hematoxylin and eosin (H&E) staining was performed according to a standard protocol. Scale bar =500 micrometers.

FIG. 6. FIG. 6 shows neutralization of Bft activity by rabbit IgG purified from anti-Bft hyper-immune sera and from pre-immunization sera, as measured by E-cadherin release from HT29 cells.

FIG. 7. FIG. 7 shows E-cadherin release from HT29 cells after treatment with the culture supernatant of NTBF or ETBF strains, plus control or anti-Bft rabbit polyclonal antibody for 18 hours. NTBF and ETBF type strains were purchased from ATCC® (Strain No. 25285 and 43859, respectively). “IBD-ETBF” refers to ETBF strains isolated from IBD patients. The dotted line represents baseline E-cadherin release without any treatment. rBft =recombinant Bft1 protein. Asterisks indicate statistical significance, as determined using the Two-way ANOVA Sidak's multiple comparison test (**** =p <0.0001).

FIG. 8. FIG. 8 shows that ETBF is enriched in subjects with active inflammatory bowel disease. Fecal samples from 152 adults with Crohn's disease or ulcerative colitis and 67 healthy controls with no history of IBD were assayed for presence of the bft toxin encoding genes by PCR. Asterisks indicate statistic difference between indicated groups as determined by Fisher's exact test (** =p<0.005).

### DETAILED DESCRIPTION

Inflammatory bowel disease (IBD), including Crohn's disease (CD) and ulcerative colitis (UC), results from dysregulated immune responses to a subset of commensal bacteria. Therapies that target this subset of bacteria hold the potential to ameliorate the disease.

B. fragilis (Bacteroides fragilis) is an anaerobic, gram-negative, rod-shaped bacterium. B. fragilis is a common commensal anaerobe (about 0.5% of the human colonic flora) that shapes the host health including the immune system. The effects of B. fragilis on the host are highly strain-dependent. Strains expressing high level of polysaccharide A have been identified to exert potent anti-inflammatory effect, whereas several other strains have been demonstrated to exert pathogenicity. Enterotoxigenic B. fragilis (ETBF) strains harboring genes encoding a pro-inflammatory enterotoxin called Bft have been identified from children and livestock with acute diarrhea! episodes. However, no causal role for ETBF in IBD has previously been established.

The present disclosure relates to the discovery that enterotoxigenic B. fragilis strains contribute to the development and progression of colitis, and may therefore be targeted in the prevention and/or treatment of inflammatory bowel diseases.

Thus, in some aspects, the disclosure provides compositions and methods for treating or preventing an inflammatory bowel disease in a subject in need thereof by administering an agent to the subject that reduces the number or pathogenic effects of a B. fragilis strain. In some aspects, the disclosure provides compositions and methods for treating or preventing an inflammatory bowel disease in a subject in need thereof, the method comprising administering to the subject an agent that reduces the number or pathogenic effects of an enterotoxigenic B. fragilis strain. The agent may be, for example, one or more of a vaccine, a passive immunotherapy (e.g., an antibody), an antibiotic, or a probiotic.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The term “microbiota,” as used herein, refers to the population of microorganisms present within or upon a subject. The microbiota of a subject includes commensal microorganisms found in the absence of disease and may also include pathobionts and disease-causing microorganisms found in subjects with or without a disease or disorder.

The term “pathobiont,” as used herein, refers to potentially disease-causing members of the microbiota that are present in the microbiota of a non-diseased or a diseased subject, and which has the potential to contribute to the development or progression of a disease or disorder.

An “effective amount” or “therapeutically effective amount” of a compound is that amount of a compound which is sufficient to provide a beneficial effect to the subject to which the compound is administered.

The terms “patient,” “subject,” “individual,” and the like are used interchangeably herein, and refer to any animal, or cells thereof, whether in vitro or in vivo, amenable to the methods described herein. In certain non-limiting embodiments, the patient, subject or individual is, by way of non-limiting examples, a human, a non-human primate, a dog, a cat, a horse, or other domestic mammal.

A “therapeutic” treatment is a treatment administered to a subject who exhibits signs or symptoms of pathology, for the purpose of diminishing or eliminating those signs or symptoms.

As used herein, “treating a disease or disorder” means reducing the severity and/or frequency with which a sign or symptom of the disease or disorder is experienced by a patient.

As used herein, the term “inflammatory bowel disease (IBD)” refers to a group of inflammatory diseases of the colon and small intestine. A number of disorders fall within the class of IBD, including Crohn's disease, ulcerative colitis, indeterminate colitis, microscopic colitis and collagenous colitis. The most common forms of IBD are Crohn's disease and ulcerative colitis. Ulcerative colitis affects the large intestine (colon) and rectum and involves the inner lining (e.g., the mucosal and sub-mucosal layer) of the intestinal wall. Crohn's disease may affect any section of the gastrointestinal tract (e.g., mouth, esophagus, stomach, small intestine, large intestine, rectum, anus, etc.) and may involve all layers of the intestinal wall. The clinical symptoms of IBD include rectal and/or intestinal bleeding, abdominal pain and cramping, diarrhea, and weight loss. In addition, IBD is a risk factor for colon cancer, and this risk for colon cancer increases significantly after eight to ten years of IBD.

The phrase “biological sample” as used herein, is intended to include any sample comprising a cell, a tissue, feces, or a bodily fluid (e.g., whole blood, plasma, or serum) in which the presence of a microbe, nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunogenic composition” refers to a composition that elicits an immunological response in a subject. The immunological response may be directed against, for example, a bacteria such as B. fragilis. In some embodiments, the immunological response is directed to a protein or other molecule produced by a bacteria, such as an enterotoxin. In some embodiments, the immunogenic composition is capable of conferring protective immunity against the bacteria and the clinical signs associated therewith.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. In some embodiments, the antibody is a synthetic antibody. The antibodies in the present disclosure may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al, 1999, Using Antibodies: A Laboratory Manual, Cold Spring Harbor Laboratory Press, NY; Harlow et al., 1989, Antibodies: A Laboratory Manual, Cold Spring Harbor, New York; Houston et al, 1988, Proc. Natl. Acad. Sci. USA 85:5879-5883; Bird et al, 1988, Science 242:423-426).

By the term “synthetic antibody” as used herein, it is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of VH (variable heavy chain immunoglobulin) genes from an animal.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a microbiota sample, tissue sample, a tumor sample, a cell or a biological fluid.

The term “adjuvant” as used herein is defined as any molecule to enhance an antigen-specific adaptive immune response.

By the term “specifically binds,” as used herein with respect to an antibody, is meant an antibody which recognizes and binds to a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine. The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “probiotic” refers to one or more bacteria that can be administered to a subject to aid in the restoration of a subject's microbiota by increasing the number of bacteria that are desired, preferred, neutral, beneficial and/or under-represented in the subject's microbiota. Similarly, a “surgical probiotic” is a strain of bacteria that is desired, preferred, neutral, beneficial and/or under-represented in the subject's microbiota and that is phylogenetically similar to a disease-associated strain of the bacteria.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, “B. fragilis” is an abbreviation for Bacteroides fragilis. B. fragilis is a gram-negative, rod-shaped bacterium, and may be characterized by its 16S RNA gene sequence (see Table 1, below). B. fragilis strains may have a 16S RNA gene sequence that is at least 95%, at least 96%, at least 97%, at least 98%, at least 99%, or 100% identical to the sequence of SEQ ID NO: 1.

TABLE 1
Exemplary B. fragilis 16S RNA gene sequence

| Species | Sequence | SEQ ID NO: |
|---|---|---|
| B. fragilis | GCGCGATTACTAGCGAATCCAGCTTCACGAAGTCGGGTTGCAGA | 1 |
| | CTTCGATCCGAACTGAGAGAGGATTTTGGGATTAGCATACGGTC | |
| | ACCCGCTAGCTGCCTTCTGTACCCCCCATTGTAACACGTGTGTA | |
| | GCCCCGGACGTAAGGGCCGTGCTGATTTGACGTCATCCCCACCT | |
| | TCCTCACATCTTACGACGGCAGTCTCTCCAGAGTCCTCAGCATG | |
| | ACCTGTTAGTAACTGAAGATAAGGGTTGCGCTCGTTATGGCACT | |
| | TAAGCCGACACCTCACGGCACGAGCTGACGACAACCATGCAGCA | |
| | CCTTCACAGCGGTGATTGCTCACTGACATGTTTCCACATCATTC | |
| | CACTGCAATTTAAGCCCGGGTAAGGTTCCTCGCGTATCATCGAA | |
| | TTAAACCACATGTTCCTCCGCTTGTGCGGGCCCCCGTCAATTCC | |
| | TTTGAGTTTCACCGTTGCCGGCGTACTCCCCAGGTGGAATACTT | |
| | AATGCTTTCGCTTGGCCGCTTACTGTATATCGCAAACAGCGAGT | |
| | ATTCATCGTTTACTGTGTGGACTACCAGGGTATCTAATCCTGTT | |
| | TGATACCCACACTTTCGAGCATCAGTGTCAGTTGCAGTCCAGTG | |
| | AGCTGCCTTCGCAATCGGAGTTCTTCGTGATATCTAAGCATTTC | |
| | ACCGCTACACCACGAATTCCGCCCACCTCTACTGTACTCAAGAC | |
| | TGACAGTATCAACTGCAATTTTACGGTTGAGCCGCARACTTTCA | |
| | CAACTGACTTACCAGTCCACCTACGCTCCCTTTAAACCCAATAA | |
| | ATCCGGATAACGCTCGGATCCTCCGTATTACCGCGGCTGCTGGC | |
| | ACGGAGTTAGCCGATCCTTATTCATATAATACATACAAAACAGT | |
| | ATACATACTGCACTTTATTCTTATATAAAAGAAGTTTACGACCC | |
| | ATAGAGCCTTCATCCTTCACGCTACTTGGCTGGTTCAGGCTAGC | |
| | GCCCATTGACCAATATTCCTCACTGCTGCCTCCCGTAGGAGTTT | |
| | GGACCGTGTCTCAGTTCCAATGTGGGGGACCTTCCTCTCAGAAC | |
| | CCCTATCCATCGAAGGCTTGGTGAGCCGTTACCTCACCAACAAC | |
| | CTAATGGAACGCATCCCCATCCTTTACCGGAATCCTTTAATAAT | |
| | GAAACCATGCGGAATCATTATGCTATCGGGTATTAATCTTTCTT | |
| | TCGAAAGGCTATCCCCGAGTAAAGGGCAGGTTGGATACGTGTTA | |
| | CTCACCCGTGCGCCGGTCGCCGGCAAAGAAAGCAAGCTTTCTT | |

As used herein, “NTBF” is an abbreviation for non-toxigenic B. fragilis, and refers to strains of B. fragilis that do not contain genes encoding and/or do not produce an enterotoxin.

As used herein, “ETBF” refers to enterotoxigenic strains of B. fragilis. ETBF strains have genes encoding a pro-inflammatory enterotoxin called Bft. ETBF strains may be differentiated from NTBF strains using several methods known to those of skill in the art, such as by using PCR to detect Bft genes in a B. fragilis sample.

As used herein, “Bft” refers to an enterotoxin produced by ETBF strains. Bft is a 20kDa metalloprotease toxin. There are three known isotypes of Bft, encoded by distinct bft loci contained within a 6 kb chromosomal region unique to ETBF strains termed the B. fragilis pathogenicity island (BfPAI). Various Bft isotypes are listed in Table 2, below. In some embodiments, an ETBF strain expresses at least one of Bftl , Bft2, and/or Bft3. Experimental studies have suggested that E-cadherin is released from cells following contact with Bft, but E-cadherin is not cleaved directly by Bft. Accordingly, E-cadherin release from cells may be used as a readout for measuring Bft activity.

TABLE 2
B. fragilis toxin (Bft) Isotypes

| Name | Sequence | SEQ ID No: |
|---|---|---|
| Bft1 | MFILNFNKMKNVKLLLMLGTAALLAACSNEADSLTTSIDAPVTASIDLQSVSYTDLATQL | 2 |
| | NDVSDFGKMIILKDNGFNRQVHVSMDKRTKIQLDNENVRLFNGRDKDSTSFILGDEFAVL | |
| | RFYRNGESISYIAYKEAQMMNEIAEFYAAPFKKTRAINEKEAFECIYDSRTRSAGKDIVS | |
| | VKINIDKAKKILNLPECDYINDYIKTPQVPHGITESQTRAVPSEPKTVYVICLRENGSTI | |
| | YPNEVSAQMQDAANSVYAVHGLKRYVNFHFVLYTTEYSCPSGDAKEGLEGFTASLKSNPK | |
| | AEGYDDQIYFLIRWGTWDNKILGMSWFNSYNVNTASDFEASGMSTTQLMYPGVMAHELGH | |
| | ILGAEHTDNSKDLMYATFTGYLSHLSEKNMDIIAKNLGWEAADGD | |
| Bft2 | MKNVKLLLMLGTAALLAACSNEADSLTTSIDTPVTASIDLQSVSYTDLATQLNDVSDFGK | 3 |
| | MIILKDNGFNRQVHVSMDKRTKIQLDNENVRLFNGRDKDSTSFILGDEFAVLRFYRNGES | |
| | ISYIAYKEAQMMNEIAEFYAAPFKKTRAINEKEAFECIYDSRTRSAGKDLVSVKINIDKA | |
| | KKILNLPECDYINDYIKTPQVPHGITESQTRAVPSEPKTVYVICLRESGSTVYPNEVSAQ | |
| | MQDAANSVYAVHGLKRFVNLHFVLYTTEYSCPSGNADEGLDGFTASLKANPKAEGYDDQI | |
| | YFLIRWGTWDNNILGISWLDSYNVNTASDFKASGMSTTQLMYPGVMAHELGHILGARHAD | |
| | DPKDLMYSKYTGYLFHLSEENMYRIAKNLGWEIADGD | |
| Bft3 | MKNVKLLLMLGTAALLAACSNEADSLTTSIDAPVTASIDLQSVSYTDLATQLNDVSDFGK | 4 |
| | MIILKDNGFNRQVHVSMDKRTKIQLDNENVRLFNGRDKDSTNFILGDEFAVLRFYRNGES | |
| | ISYIAYKEAQMMNEIAEFYAAPFKKTRAINEKEAFECIYDSRTRSAGKYPVSVKINVDKA | |
| | KKILNLPECDYINDYIKTPQVPHGITESQTRAVPSEPKTVYVICLRENGSTVYPNEVSAQ | |
| | MQDAANSVYAVHGLKRYVNLHFVLYTTEYACPSGNADEGLDGFTASLKANPKAEGYDDQI | |
| | YFLIRWGTWDNNILGISWLNSYNVNTASDFKASGMSTTQLMYPGVMAHELGHILGANHAD | |
| | DPKDLMYSKYTGYLFHLSEKNMDIIAKNLGWEIADGD | |

A Bft described herein may have a sequence of any one of SEQ ID NO: 2-4. In some embodiments, the Bft may have a sequence that is at least 95%, at least 96%, at least 97%, at least 98%, or at least 99% identical to the sequence of any one of SEQ ID NO: 2-4.

As used herein, “pro-inflammatory cytokine” refers to a signaling molecule that promotes inflammation. Non-limiting examples of pro-inflammatory cytokines include interleukin-1 (IL-1), interleukin-3 (IL-3), interleukin-6 (IL-6), interleukin-12 (IL-12), interleukin-17 (IL-17), interleukin-18 (IL-18), tumor necrosis factor (TNF), interferon gamma (IFN-gamma), macrophage inflammatory protein 2 (MIP-2), RANTES (CCLS) and granulocyte-macrophage colony stimulating factor (GM-CSF).

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Immunogenic Compositions (e.g., Vaccines)

Provided herein are immunogenic compositions that can reduce the number or pathogenic effects of one or more bacteria associated with the development or progression of IBD. In some embodiments, the immunogenic composition may reduce the number or pathogenic effects of a B. fragilis strain, for example an enterotoxigenic B. fragilis strain. In some embodiments, the immunogenic composition may reduce the effects of an enterotoxin (e.g., Bft1, Bft2, and/or Bft3) produced by a B. fragilis strain.

In some embodiments, immunogenic compositions may reduce the levels of one or more pro-inflammatory cytokines in a subject. For example, an immunogenic composition may reduce levels of IL-1, IL-3, IL-6, IL-12, IL-17, IL-18, TNF, IFN-gamma, MIP-2, RANTES and/or GM-CSF. In some embodiments, an immunogenic composition reduces levels of one or both of TNF and IL-17 in a subject.

One type of immunogenic composition encompassed by the disclosure is a vaccine. The terms “vaccine” and “vaccine composition” are used interchangeably herein.

In some embodiments, the vaccine comprises at least one bacterium. For example, in some embodiments, the vaccine comprises an inactivated or killed bacterium. Inactivated or killed indicates the bacterium has lost the ability to cause disease in mammals but retains an immunogenic property thereof, particularly the ability to generate a specific immune response against one or more antigens of the bacterium. The term inactivated bacterium also includes non-virulent bacterium. Methods for preparing or selecting inactivated bacteria are well known in the art. They include heat-inactivation methods, or chemical inactivation methods. Inactivation may be carried out by exposing the bacterium to a chemical agent such as formalin, formaldehyde, paraformaldehyde, β-propiolactone, ethyleneimine, binary ethyleneimine (BEI), thimerosal, or derivatives thereof. Alternatively, inactivation may be carried out by physical treatments such as heat treatment or sonication. The inactivated pathogen may be concentrated by conventional concentration techniques, in particular by ultrafiltration, and/or purified by conventional purification means, in particular using chromatography techniques including but not limited to gel-filtration, ultracentrifugation on a sucrose gradient, or selective precipitations.

In some embodiments, an inactivated or killed bacterium of the vaccine composition is a Segmented Filamentous Bacteria (SFB) or Helicobacter flexispira, or a bacteria from at least one family selected from the group consisting of Lactobacillus, Helicobacter, S24-7, Erysipelotrichaceae and Prevotellaceae. In some embodiments, the bacteria from the family Prevotellaceae is a bacteria from the genera of Paraprevotella or Prevotella. In other embodiments, the bacteria is selected from Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, and Weissella.

In some embodiments, the inactivated or killed bacterium of the vaccine composition is a B. fragilis. For example, the inactivated or killed bacterium of the vaccine composition may be an enterotoxigenic strain of B. fragilis. Exemplary ETBF strains that may be used in the vaccine composition include 86-5443-2-2, 2-078382-3 (ATCC® No. 43858), BOB25, 20656-2-1, 20793-3, 20793-3, 20656-2-1, 8B-5443-2-2, and ATCC® No. 43859. In some embodiments, an ETBF strain for use in a vaccine composition may be isolated from a human fecal sample. In some embodiments, an ETBF strain may be an engineered strain, such as a non-toxigenic B. fragilis strain engineered to express or overexpress BFT.

In some embodiments, the vaccine comprises an antigen (e.g., a peptide or polypeptide), a nucleic acid encoding an antigen (e.g., an antigen expression vector), or a cell expressing or presenting an antigen or cellular component. For example, in some embodiments, the antigen is an antigen of one or more bacteria associated with the development or progression of a disease or disorder, thereby inducing an immune response against the one or more bacteria.

In some embodiments, the antigen is a toxin produced by a bacteria, such as an enterotoxin. In some embodiments, the antigen is an enterotoxin (e.g., an inactivated enterotoxin) produced by a B. fragilis strain, or a fragment thereof. The enterotoxin may comprise, for example, the sequence of any one of SEQ ID NO: 2-4. In some embodiments, the enterotoxin has a sequence that is about 90%, about 95%, about 96%, about 97%, about 98%, about 99% identical to the sequence of any one of SEQ ID NO: 2-4. In some embodiments, the enterotoxin is a peptide fragment, having a sequence isolated or derived from any one of SEQ ID NO: 2-4. In some embodiments, the antigen (e.g., an inactivated B. fragilis enterotoxin) is recombinant.

In some embodiments, the vaccine comprises a mixture that comprises an additional immunostimulatory agent, or one or more nucleic acids encoding an immunostimulatory agent. Immunostimulatory agents include but are not limited to an antigen, an immunomodulator, an antigen presenting cell or an adjuvant. An adjuvant refers to a compound that enhances the immune response when administered together (or successively) with the immunological composition. Examples of suitable adjuvants include cholera toxin, E. coli heat-labile toxin, E. coli enterotoxin, salmonella toxin, alum, nanoparticle-based adjuvants, a-interferon(IFN-α), β-interferon (IFN-β), γ-interferon, platelet derived growth factor (PDGF), TNFa, TNFp, GM-CSF, epidermal growth factor (EGF), cutaneous T cell-attracting chemokine (CTACK), epithelial thymus-expressed chemokine (TECK), mucosae-associated epithelial chemokine (MEC), IL-1, IL-2, IL-4, IL-5, IL-6, IL-10, IL-12, IL-18, MHC, CD80, and CD86. Further examples of suitable adjuvants and/or immunomodulators include, but are not limited to, complete or incomplete Freund's adjuvant, RIBI (e.g., muramyl dipeptides, etc.), KLH peptide, cholera toxin or a portion thereof, salmonella toxin or a portion thereof, E. coli heat labile enterotoxin or a portion thereof, E. coli enterotoxin or a portion thereof, AB5 toxins or a portion thereof, mineral salts, aluminum salts (e.g., hydroxide, phosphate, Alum, etc.), calcium phosphate, liposomes, virosomes (unilamellar liposomal vehicles, immunostimulating reconstituted influenza virosomes [IRIV]), virus-like particles, cochleates, eurocine (e.g., monoglycerides with fatty acids, etc.), archaeal lipids, ISCOMS (e.g., immunostimulating complexes, structured complex of saponins and lipids, etc.), microparticles (e.g., PLG, etc.), emulsions (e.g., MF59, Montanides, etc.), monophosphoryl lipid (MPL) or synthetic derivatives, N-acetyl-muramyl-L-alanyl-D-isoglutamine (MDP) or a derivative, Detox (MPL +CWS), ASO4 (Alum +MPL), AS02 (oil- in-water emulsion +MPL +QS21), AS01 (liposomes +MPL +QS21), OM-174 (e.g., Lipid A derivative, E. coli, etc.), OM-triacyl, oligonucleotides (e.g., CpG, etc.), double-stranded R A (dsR A), pathogen-associated molecular patterns (PAMPs), TLR ligands (e.g., flagellin, monophosphoryl lipid A, etc.), saponins (e.g., Quils, QS-21, etc.), chitosan, a-galactosylceramide, small-molecule immune potentiators (SMIPs) (e.g., imiquimod, resiquimod [R848], etc.), a cytokine or chemokine (e.g., IL-2, IL-12, GM-CSF, Flt3, etc.), an accessory molecule (e.g., B7.1, etc.), liposomes (e.g., DNPC/Chol, etc.), DC Choi (e.g., lipoidal immunomodulators able to self-organize into liposomes, etc.), nanoparticle-based adjuvants, PLA (polylactic acid) microparticles, PLG (poly[lactide-co-glycolide]) microparticles, Poly(DL-lactide-co-glycolide) microparticles, polystyrene (latex) microparticles, proteosomes (e.g., hydrophobic, proteinaceous, nanoparticles comprised of purified N. meningitidis outer membrane proteins, etc.), and 3′,5′-Cyclic diguanylic acid (c-di-GMP). Such example adjuvants and/or immunomodulators, as well as others, are readily described in available literature, and are useful in the compositions and methods of the disclosure.

Furthermore, a vaccine of this disclosure may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such.

Vaccine administration may be performed once (i.e., by a single administration) or more than once (i.e., by multiple administrations).

The vaccine composition can further comprise other agents for formulation purposes according to the mode of administration to be used. In cases where pharmaceutical vaccine compositions are injectable, they may be sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can include vehicles, adjuvants, carriers, or diluents.

The vaccine, or other immunological composition, can be formulated for administration systemically or locally by many different routes including orally, parenterally, sublingually, transdermally, rectally, transmucosally, topically, via inhalation, via buccal administration, intrapleurally, intravenously, intraarterially, intragastrically, nasally, intraperitoneally, subcutaneously, intramuscularly, intranasally intrathecally, and intraarticularly or combinations thereof.

In some embodiments, the vaccine may reduce the levels of one or more pro-inflammatory cytokines in a subject. For example, the vaccine may reduce levels of IL-1, IL-3, IL-6, IL-12, IL-17, IL-18, TNF, IFN-gamma, MIP-2, RANTES and/or GM-CSF in a subject. In some embodiments, a vaccine reduces levels of one or both of TNF and IL-17 in a subject.

### Passive Immunotherapy

Also provided herein are passive immunotherapies, and compositions comprising the same, for treating and/or preventing IBD. In embodiments, an inflammatory bowel disease in a subject may be treated by administering to the subject a therapeutically effective amount of a passive immunotherapy or passive vaccine.

The passive immunotherapy or passive vaccine can be administered systemically or locally by many different routes including orally, parenterally, sublingually, transdermally, rectally, transmucosally, topically, via inhalation, via buccal administration, intrapleurally, intravenously, intraarterially, intragastrically, nasally, intraperitoneally, subcutaneously, intramuscularly, intranasally, intrathecally, and intraarticulally or combinations thereof. Alternatively, the passive immunotherapy or passive vaccine can be administered rectally or by enema.

In some embodiments, the passive immunotherapy or passive vaccine reduces the number or pathogenic effects of at least one type (e.g., genus, species, strain, sub-strain, etc.) of bacteria, such as a Segmented Filamentous Bacteria (SFB) or Helicobacter flexispira, or a bacteria from at least one family selected from the group consisting of Lactobacillus, Helicobacter, S24-7, Erysipelotrichaceae and Prevotellaceae. In some embodiments, the bacteria from the family Prevotellaceae is a bacteria from the genera of Paraprevotella or Prevotella. In some embodiments, the bacteria is selected from Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainf uenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacteriurn spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, and Weissella.

In some embodiments, a passive immunotherapy or passive vaccine reduces the number or pathogenic effects of a B. fragilis strain (e.g., an enterotoxigenic B. Fragilis strain). In some embodiments, a passive immunotherapy or passive vaccine response reduces the number or pathogenic effects of an enterotoxin, such as an enterotoxin produced by a B. fragilis strain (e.g., Bft1, Bft2, and/or Bft3).

In some embodiments, a passive immunotherapy or passive vaccine comprises an antibody. Generally, an antibody comprises two heavy chains linked to each other by disulfide bonds and two heavy chains linked to light chains by disulfide bonds. There are two types of light chains, lambda and kappa. There are five main heavy chain classes (or iso types) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The term “antibody” includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BITE (Bispecific T-cell Engager, scFv-scFv tandems to attract T cells); DVD-Ig (dual variable domain antibody, bispecific format); SIP (small immunoprotein, a kind of minibody); SM IP (“small modular immunopharmaceutical” scFv-Fc dimer; DART (ds-stabilized diabody “Dual Affinity ReTargeting”); small antibody mimetics comprising one or more CDRs and the like. The term “antibody” may refer to either polyclonal or monoclonal antibodies.

Techniques for preparing and using various antibody-based constructs and fragments are well known in the art (see Kabat et al, 1991, specifically incorporated herein by reference). Diabodies, in particular, are further described in EP 404,097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al, 2004; Reff & Heard, 2001; Reiter et al., 1996; and Young et al., 1995 further describe the production of effective antibody fragments. In some embodiments, the antibody is a “chimeric” antibody, for example as described in U.S. Pat. No. 4,816,567. In some embodiments, the antibody is a humanized antibody, for example as described U.S. Pat. Nos. 6,982,321 and 7,087,409. In some embodiments, the antibody is a human antibody, for example as described in U.S. Pat. Nos. 6,075,181 and 6,150,584. In some embodiments, the antibody is a single domain antibody, for example as described in EP 0 368 684, WO 06/030220 and WO 06/003388.

In embodiments, the antibody binds to a B. fragilis (e.g., an enterotoxigenic B. fragilis). In some embodiments, the antibody binds to an enterotoxin produced by a B. Fragilis, such as Bft1, Bft2, and/or Bft3. Exemplary antibodies that bind to one or more of Bft1, Bft2, and Bft3 are described in Mootien, S., et al., PLoS One, Vol. 12, Issue 3 (2017); and Qadri, F., et al., Clin. Diagn. Lab. Immunol., Vol. 3, Issue 5, (1996), which are incorporated by reference herein in their entireties. In some embodiments, the antibody is a monoclonal antibody. In some embodiments, the antibody is humanized. In some embodiments, the antibody is an IgM, IgD, IgG, IgA or IgE. In some embodiments, the antibody is monoclonal anti-Bft antibody ICT11 (see Qadri, et al.)

In some embodiments, the antibody may reduce the levels of one or more pro-inflammatory cytokines in a subject. For example, the antibody may reduce levels of IL-1, IL-3, IL-6, IL-12, IL-17, IL-18, TNF, IFN-gamma, MIP-2, RANTES and/or GM-CSF. In some embodiments, an antibody reduces levels of one or both of TNF and IL-17 in a subject.

### Antibiotics

In embodiments, a therapeutically effective amount of antibiotic composition comprising an effective amount of at least one antibiotic, or a combination of several types of antibiotics, may be administered to the subject. The antibiotic composition may be administered alone, or in combination with an antibody or a vaccine of the disclosure.

In some embodiments, the administered antibiotic reduces the number or pathogenic effects of at least one type (e.g., genus, species, strain, sub-strain, etc.) of bacteria. In some embodiments, the at least one type (e.g., genus, species, strain, sub-strain, etc.) of bacteria is selected from a Segmented Filamentous Bacteria (SFB) or Helicobacter flexispira, or a bacteria from at least one family selected from the group consisting of Lactobacillus, Helicobacter, S24-7, Erysipelotrichaceae and Prevotellaceae. In some embodiments, the bacteria from the family Prevotellaceae is a bacteria from the genera of Paraprevotella or Prevotella. In some embodiments, the bacteria is selected from Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Bamesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfiuenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., OsciUospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar, and Weissella. In some embodiments, the administered antibiotic reduces the number or pathogenic effects of a B. fragilis. In some embodiments, the administered antibiotic reduces the number or pathogenic effects of an enterotoxigenic B. fragilis.

The type and dosage of the administered antibiotic will vary widely, depending upon the nature of the inflammatory disease or disorder, the character of the subject's altered microbiota, the subject's medical history, the frequency of administration, the manner of administration, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some embodiments, the administered antibiotic is at least one of lipopeptide, fluoroquinolone, ketolide, cephalosporin, amikacin, gentamicin, kanamycin, neomycin, netilmicin, paromomycin, streptomycin, tobramycin, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpodoxime, cefteram, ceftibuten, ceftiofur, ceftiolene, ceftizoxime, ceftriaxone, cefoperazone, ceftazidime, cefclidine, cefepime cefluprenam, cefoselis, cefozopran, cefpirome, cefquinome, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftaroline, ceftioxide, cefuracetime, imipenem, primaxin, doripenem, meropenem, ertapenem, flumequine, nalidixic acid, oxolinic acid, piromidic acid pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, azithromycin, erythromycin, clarithromycin, dirithromycin, roxithromycin, telithromycin, amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, ticarcillin, sulfamethizole, sulfamethoxazole, sulfisoxazole, trimethoprim-sulfamethoxazole, demeclocycline, doxycycline, minocycline, oxytetracycline, tetracycline, linezolid, clindamycin, metronidazole, vancomycin, vancocin, mycobutin, rifampin, nitrofurantoin, chloramphenicol, or derivatives thereof.

### Probiotics

In some embodiments, a therapeutically effective amount of a probiotic composition may be administered to the subject. The probiotic composition may be administered alone, or in combination with an immunogenic composition (e.g. a vaccine) of the disclosure.

In some embodiments, the probiotic composition comprises an effective amount of at least one type (e.g., genus, species, strain, sub-strain, etc.) of bacteria, or a combination of several types of bacteria. In some embodiments, the probiotic is a surgical probiotic. In some embodiments, the disclosure is a method of treating an inflammatory bowel disease or disorder of a subject in need thereof, including the step of administering to the subject at least one type (e.g., genus, species, strain, sub-strain, etc.) of bacteria, or a combination of several types of bacteria, that is desired, preferred, neutral, beneficial, and/or under-represented in the subject's microbiota.

In some embodiments, the at least one type of bacteria is at least one bacterium of a first strain of a species of bacteria, wherein the first strain of the species of bacteria does not contribute to the development or progression of inflammatory bowel disease in the subject, and wherein the species of bacteria comprises at least a second strain of bacteria, and wherein the second strain of the species of bacteria does contribute to the development or progression of the inflammatory bowel disease.

In some embodiments, the at least one type of bacteria is at least one bacterium of a species of bacteria identified from a healthy subject that does not have IBD.

Bacteria administered according to the methods of the present disclosure can comprise live bacteria. One or several different types of bacteria can be administered concurrently or sequentially. Such bacteria can be obtained from any source, including being isolated from a microbiota and grown in culture using known techniques.

In some embodiments, the administered bacteria used in the methods of the disclosure further comprise a buffering agent. Examples of useful buffering agents include sodium bicarbonate, milk, yogurt, infant formula, and other dairy products.

Administration of a bacterium can be accomplished by any method suitable for introducing the organisms into the desired location. The bacteria can be mixed with a carrier and (for easier delivery to the digestive tract) applied to a liquid or to food. The carrier material should be non-toxic to the bacteria as wells as the subject. Preferably, the carrier contains an ingredient that promotes viability of the bacteria during storage. The formulation can include added ingredients to improve palatability, improve shelf-life, impart nutritional benefits, and the like. The dosage of the administered bacteria (e.g., probiotic, surgical probiotic) will vary widely, depending upon the nature of the inflammatory disease or disorder, the character of the subject's altered microbiota, the subject's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve colonization of the gastrointestinal tract with the desired bacteria. In some embodiments, the dose ranges from about 10⁶ to about 10¹⁰ CFU per administration, or about 10¹⁰ to about 10¹³ CFU per administration. In some embodiments, the dose ranges from about 10⁴ to about 10⁶ CFU per administration.

In some embodiments, the present disclosure relates to a method comprising administering to a subject in need of such treatment, an effective amount of at least one gastric, esophageal, or intestinal bacterium, or combinations thereof. In some embodiments, the bacteria are administered orally. Alternatively, bacteria can be administered rectally or by enema.

One of the organisms contemplated for administration to modify the altered microbiota is at least one Lactobacillus spp. In some embodiments, the bacteria administered in the therapeutic methods of the disclosure comprise administration of a combination of organisms.

While it is possible to administer a bacteria for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Although there are no physical limitations to delivery of the probiotic formulations of the present disclosure, oral delivery is preferred for delivery to the digestive tract because of its ease and convenience, and because oral formulations readily accommodate additional mixtures, such as milk, yogurt, and infant formula. For delivery to colon, bacteria can be also administered rectally or by enema.

In some embodiments, treatment of IBD is achieved by both administering at least one type (e.g., genus, species, strain, sub-strain, etc.) of bacteria to supplement the numbers of at least one type (e.g., genus, species, strain, sub-strain, etc.) of bacteria that is under-represented in the subject, and administering at least one antibiotic to reduce the numbers of at least one type (e.g., genus, species, strain, sub-strain, etc.) of bacteria that is over-represented in the subject.

### Methods of Treatment

Also provided herein are methods of treating or preventing an inflammatory bowel disease (e.g., Crohn's disease or ulcerative colitis) in a subject in need thereof. In some embodiments, a method for treating or preventing an inflammatory bowel disease in a subject comprises administering to the subject an agent to reduce the number or pathogenic effects of a B. fragilis strain, such as an enterotoxigenic B. fragilis strain, wherein the agent comprises, for example, a vaccine or an antibody. In some embodiments, a method for treating or preventing an inflammatory bowel disease in a subject comprises administering to the subject an agent to reduce the effects of an enterotoxin produced by a B. fragilis strain, wherein the agent comprises, for example, a vaccine or an antibody. In some embodiments, the method for treating or preventing an inflammatory bowel disease in a subject comprises administering a vaccine composition to the subject.

In some embodiments, a method of treating a subject diagnosed with IBD comprises administering to the subject an agent to reduce the number or pathogenic effects of a B. fragilis strain, wherein the subject is diagnosed with IBD by detecting the presence of the B. fragilis strain in a biological sample of the patient. In some embodiments, a method of treating a subject diagnosed with IBD comprises administering to the subject an agent to reduce the expression or activity of a B. fragilis toxin, wherein the subject is diagnosed with IBD by detecting the presence of the B. fragilis toxin in a biological sample of the patient. In some embodiments, the subject is diagnosed with IBD before administration of the agent. In some embodiments, the subject is diagnosed with IBD after administration of the agent.

In some embodiments, a method of treating a subject in need thereof comprises detecting the presence of a B. fragilis strain in a biological sample of the subject, and administering to the subject an agent to reduce the number of or pathogenic effects of the B. fragilis strain. In some embodiments, a method of treating a subject in need thereof comprises detecting the presence of the B. fragilis toxin in a biological sample of the subject, and administering to the subject an agent to reduce the expression or activity of the B. fragilis toxin. In some embodiments, detecting the presence of a B. fragilis strain or toxin is performed before administering an agent. In some embodiments, detecting the presence of a B. fragilis strain or toxin is performed after administering the agent. In some embodiments, detecting the presence of a B. fragilis strain or toxin is performed both before and after administering the agent.

The biological sample for use in the methods described above may be, for example, a stool sample or a blood sample (e.g., a whole blood, plasma, or serum sample). In some embodiments, the biological sample may be a cell, a tissue, or a bodily fluid. In some embodiments, the biological sample may obtained through a biopsy.

The presence of a B. fragilis strain or a B. fragilis toxin may be detected using any acceptable method. For example, the presence of a B. fragilis strain may be detected using 16sRNA gene sequencing (see, e.g., Kozich et al., Applied and Environmental Microbiology, 79(17), 5112-5120 (2013)). In some embodiments, detection of a B. fragilis 16sRNA gene (e.g., SEQ ID NO: 1 or sequence at least 95% identical thereto) in a sample (e.g., a fecal sample) indicates the presence of a B. fragilis strain in that sample.

A B. fragilis toxin may be detected, for example, using a PCR-based approach. In PCR-based approaches, DNA may be extracted from a patient stool sample (or other patient sample), and a DNA region of interest may be amplified using appropriate primers. When detecting Bft, one or more of the following exemplary primer pairs may be used: SEQ ID NO: 5 and 6, SEQ ID NO: 7 and 8, and SEQ ID NO: 9 and 10. The presence of an amplified product may then be visualized using gel electrophoresis. Exemplary PCR-based assays for detecting Bft are also described in Boleij et al., Clinical Infectious Diseases: an Official Publication of the Infectious Diseases Society of America, 60(2), 208-215, 2015; Odamaki et al., Anaerobe, 18(1), 14-18, 2012; and Franco et al., Molecular Microbiology, 45(4), 1067-1077, 2002, which are each incorporated by reference herein in their entireties.

A B. fragilis toxin may also be detected using non-PCR based approaches. In some embodiments, a blood sample may be tested for the presence of anti-Bft antibodies, or for soluble toxin. In some embodiments, a B. fragilis toxin may be detected using an ELISA. Exemplary ELISA-based approaches for determining the presence of a B. fragilis toxin are provided in Mootien, S., et al., PLoS One, Vol. 12, Issue 3 (2017); and Qadri, F., et al., Clin. Diagn. Lab. Immunol., Vol. 3, Issue 5, (1996), which are each incorporated by reference herein in their entireties.

In some embodiments, the agent administered to the patient may bind and/or inhibit at least one of Bft1, Bft2, and Bft3. In some embodiments, the agent binds and/or inhibits Bft1 and Bft2. In some embodiments, the agent binds and/or inhibits Bft1 and Bft3. In some embodiments, the agent binds and/or inhibits Bft2 and Bft3. In some embodiments, the agent binds and/or inhibits Bft1, Bft2, and Bft3.

In some embodiments, the agent may bind and/or inhibit Bft that is bound to a cell membrane. In some embodiments, the agent may bind and/or inhibit secreted Bft. In some embodiments, the agent may bind and/or inhibit intracellular Bft.

In some embodiments, the agent may decrease Bft activity by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In some embodiments, the agent may decrease Bft activity by about 5% to about 25%, about 25% to about 50%, about 50% to about 75%, or about 75% to 100%. In some embodiments, the agent may decrease Bft activity by about 95% to 100%, e.g., a decrease in activity of 95%, 96%, 97%, 98%, 99%, or 100%. E-cadherin release may be used as a readout of Bft activity. Thus, in come embodiments, the agent may decrease E-cadherin release by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In some embodiments, the agent administered to the subject is an antibody. In some embodiments, the antibody is a monoclonal antibody. In some embodiments, the antibody is a humanized antibody. The antibody may bind to, for example, an enterotoxigenic B. fragilis, or to an enterotoxin produced thereby. In some embodiments, the antibody binds to at least one of Bft1, Bft2, and Bft3.

In some embodiments, the agent administered to the subject is a vaccine. In embodiments, the vaccine comprises an inactivated B. fragilis, such as an enterotoxigenic B. fragilis. In some embodiments, the vaccine comprises a B. fragilis enterotoxin. The B. fragilis enterotoxin may comprise the amino acid sequence of any one of SEQ ID NO: 2-4, or a sequence at least 95%, at least 96%, at least 97%, at least 98%, or at least 99% identical thereto. In some embodiments, the B. fragilis enterotoxin is recombinant.

The agent may be administered in a pharmaceutical composition. In some embodiments, the pharmaceutical composition comprises an adjuvant, and/or a pharmaceutically acceptable carrier.

In some embodiments, the agent is administered orally or intramuscularly to the subject. In some embodiments, the agent is administered orally, parenterally, sublingually, transdermally, rectally, transmucosally, topically, via inhalation, via buccal administration, intrapleurally, intravenously, intraarterially, intragastrically, nasally, intraperitoneally, subcutaneously, intramuscularly, intranasally, intrathecally, or intraarticularly, or combinations thereof. The agent may be administered once, or more than once to the subject.

In some embodiments, more than one agent is administered to the subject. For example, more than one antibody, more than one vaccine, more than one antibiotic and/or more than one probiotic may be administered to the subject. In some embodiments, a first agent and a second agent are administered to a subject, wherein each of the first agent and the second agent are independently selected from an antibody, a vaccine, an antibiotic, and a probiotic. In some embodiments, the first agent and the second agent may be administered simultaneously. In some embodiment, the first agent may be administered before the second agent. In some embodiments, the first agent and the second agent are administered at therapeutically effective intervals.

In some embodiments, the subject is a mammal. In some embodiments, the subject is a human, a non-human primate, a dog, a cat, a horse, or other domestic mammal. In some embodiments, the subject is a human. The subject may be a male or a female. In some embodiments, the subject is a least 18 years of age. In some embodiments, the subject is less than 18 years of age. In some embodiments, the subject is less than 5 years of age. In some embodiments, the subject is less than 1 year of age.

In some embodiments of the methods of the disclosure, the subject has “active IBD.” Active IBD can include symptoms such as bloody diarrhea, abdominal pain, and fever. In some embodiments, the subject has “inactive IBD.” Inactive IBD may be associated with minimal to no intestinal inflammation and a lack of severe gastrointestinal illness. Whether an IBD patient has “active” or “inactive” IBD may be determined using the physician administered activity indexes Simple Clinical Colitis Activity Index (SCCAI) (Walmsley, et al., Gut, 43(1), 29-32, 1998) for subjects with ulcerative and indeterminate colitis and Harvey Bradshaw Index (HBI) (Harvey & Bradshaw, Lancet, 1(8167), 514, 1980) subjects with Crohn's disease. Subjects with a score >4 are considered to have active disease.

In some embodiments, the treatment reduces the levels of one or more pro-inflammatory cytokines in a subject. A decrease in pro-inflammatory cytokines may be detected in a blood sample, in a fecal sample, or in a biopsy sample. In some embodiments, the treatment may reduce levels of IL-1, IL-3, IL-6, IL-12, IL-17, IL-18, TNF, IFN-gamma, MIP-2, RANTES and/or GM-CSF. In some embodiments, the treatment reduces levels of one or both of TNF and IL-17 in a subject.

Without further description, it is believed that one of ordinary skill in the art can, using the preceding description and the following illustrative examples, make and utilize the compositions of the present disclosure and practice the claimed methods. The following working examples therefore specifically point out the preferred embodiments, and are not to be construed as limiting in any way the remainder of the disclosure.

### EXAMPLES

The following examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

### Example 1

ETBF strains isolated from IBD patients cause cecal inflammation in mice

Germ free mice (N=3-4) were colonized with B. fragilis non-toxigenic (NTBF) type strain ATCC® 25285, or one of four ETBF strains isolated from different IBD subjects. On day 4 after colonization, mice were sacrificed and examined for signs of intestinal inflammation.

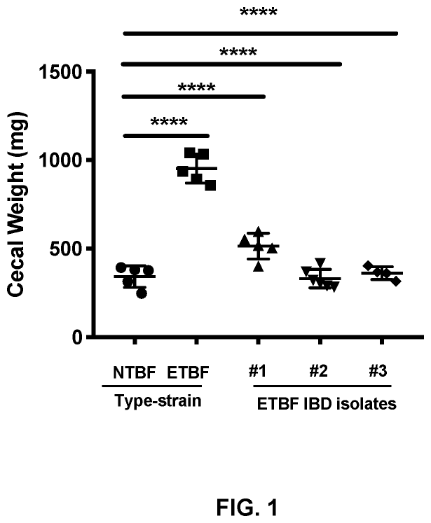

Colonization of the germ-free mice with ETBF strains isolated from IBD patients resulted in spontaneous cecal inflammation. In contrast to colonization with NTBF, colonization with each ETBF strain tested resulted in a significant decrease in cecum weight (FIG. 1), a marker for cecal injury and inflammation associated with ETBF strains (See Rhee, K. J., et al., Infection and Immunity, 77(4), 1708-1718).

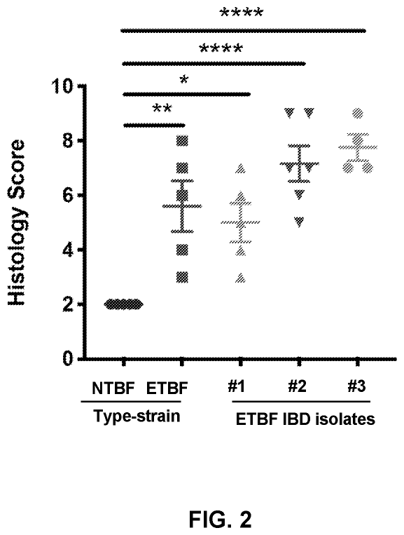

In a separate experiment, germ-free mice (N=5-6) were colonized with NTBF- or ETBF-type strains, or ETBF strains isolated from IBD patients. At day 3 post-colonization, mice were sacrificed and cecal tissue was sectioned. H&E stained sections were examined and assigned a histological score. As shown in FIG. 2, little to no inflammation was observed in mice colonized with a NTBF-type strain. In contrast, mice colonized with an ETBF-type strain, or an ETBF strain isolated from an IBD patient had high levels of inflammation. These differences were statistically significant.

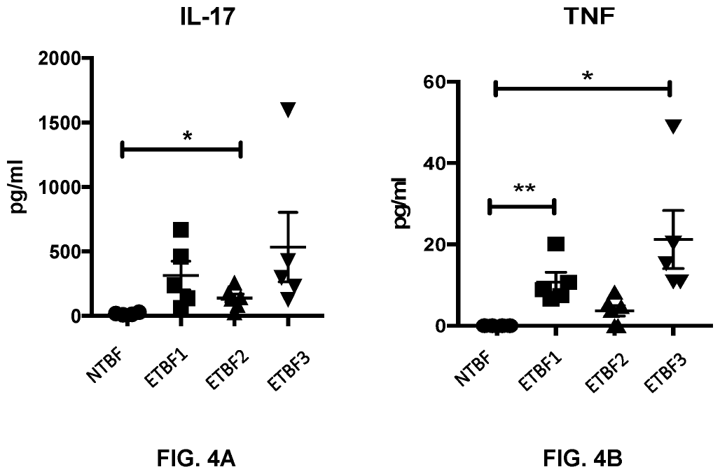

The explanted cecal tissue was also tested for the presence of pro-inflammatory cytokines IL-17 and TNF. As shown in FIG. 4A, IL-17 levels were elevated in mice colonized with ETBF strains isolated from IBD patients, as compared to mice colonized with an NTBF strain. TNF levels were also elevated in the ETBF-colonized mice (FIG. 4B).

To determine if the ETBF strains isolated from IBD patients also cause inflammation in various microbiota background settings, germ free mice were colonized with ETBF alone (monocolonization), in combination with a 9-strain synthetic microbiota community (synthetic microbiota), in combination with patient microbiota sample (patient microbiota) or in combination with a microbiota sample from a healthy patient (healthy microbiota). Mice colonized with NTBF were used as a control.

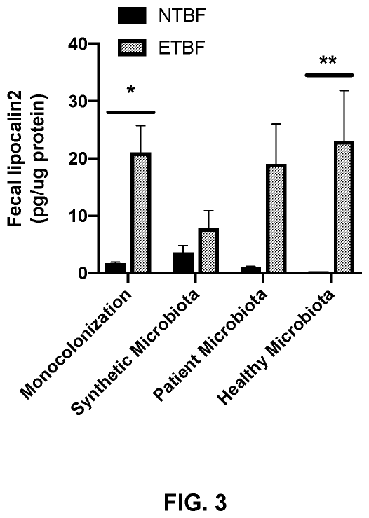

Five days after colonization, intestinal inflammation was examined by measuring fecal lipocalin2 levels. As shown in FIG. 3, ETBF strains from IBD patients induced intestinal inflammation under monocolonization, in the 9-strain synthetic microbiota community, in fecal microbiota of IBD patients (i.e., the original source of the ETBF strains used herein), and in total fecal microbiota from a healthy person.

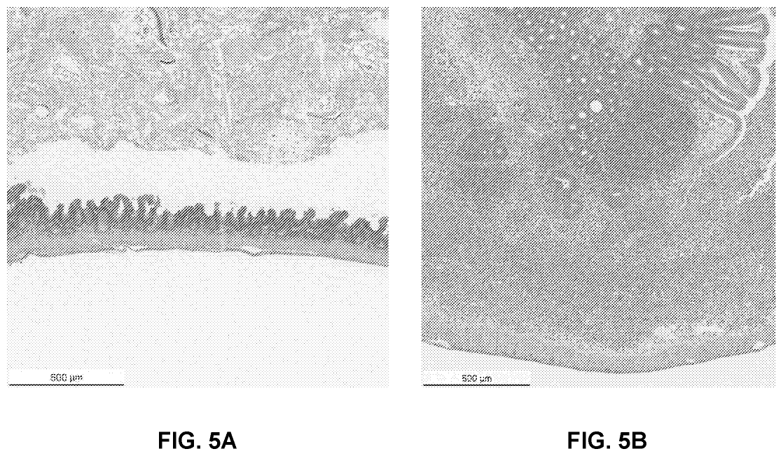

The ability of ETBF to cause inflammation was also confirmed by histology. FIG. 5A and 5B are representative histology images of cecal tissues from mice colonized with NTBF or ETBF (IBD isolate) strains, in the context of a 9-strain synthetic microbiota background. In FIG. 5B, it is evident that ETBF colonization lead to dramatically increased levels of inflammation in these tissues.

Taken together, these data show that at physiologically relevant colonization abundance, ETBF can exert potent pathogenicity to the host, resulting in IBD-like disease in mice. Thus, these data demonstrate a causal role for ETBF in IBD pathogenesis.

### Example 2

Human IBD patients colonized with ETBF are more likely to have active disease IBD patients were recruited at outpatient clinics. After the patients provided informed consent, they were asked to provide clinical information as well as a stool sample. DNA was extracted from ˜25-75 mg of fecal sample and 16s rRNA gene sequencing was used to identify subjects that carried B. fragilis at a relative abundance >0.05%. The presence of the bft toxin gene was subsequently assayed in these B. fragilis positive subjects N=152 IBD and N=67 controls using PCR with three primer sets (Table 3) and gel electrophoresis. Subjects where considered ETBF positive if two or more of the primer sets produced a band of the expected size.

TABLE 3
Primers used to detect bft

| | | Amplification | SEQ |
|---|---|---|---|
| | | Product Size | ID |
| Name | Sequence (5′ to 3′) | (base pairs) | NO: |
| 368-F | GAACCTAAAACGGTATATGT | 368 | 5 |
| 368-R | GTTGTAGACATCCCACTGGC | | 6 |
| Bft-F | GGATACATCAGCTGGGTTGTAG | 296 | 7 |
| Bft-R | GCGAACTCGGTTTATGCAGT | | 8 |
| BFTF | CGCGGCATTATTAGCTGCATGTTC | 991 | 9 |
| | TAATG | | |
| P4 | GATACATCAGCTGGGTTGTAGACA | | 10 |
| | TCCCA | | |

The disease activity of IBD subjects was determined using the physician administered activity indexes Simple Clinical Colitis Activity Index (SCCAI) (Walmsley, et. al. Gut, 43(1), 29-32, 1998) for subjects with ulcerative and indeterminate colitis and Harvey Bradshaw Index (HBI) (Harvey & Bradshaw, Lancet, 1(8167), 514, 1980) subjects with Crohn's disease. Subjects with a score >4 are considered to have active disease.

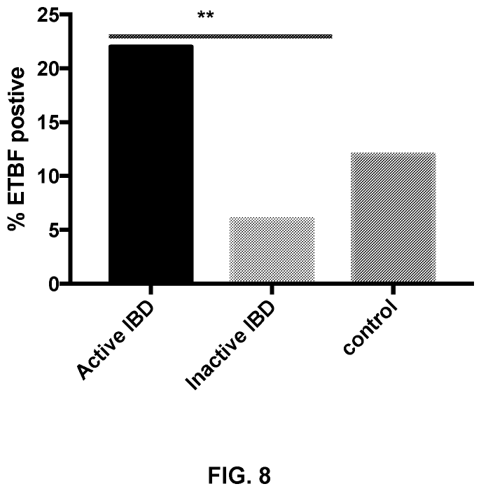

As shown in FIG. 8, about 10% of the healthy control patients (7 out of 67) tested positive for ETBF. About 6% (7 out of 116) of patients with inactive IBD, and about 22% (8 out of 36) of patients with active IBD tested positive for ETBF. This data demonstrates that IBD patients colonized with ETBF are more likely to have the active form of the disease.

### Example 3

Antibodies that Target and Inhibit Bft

Bft, the toxin produced by ETBF, is responsible for ETBF's pathogenic activity. Accordingly, inhibition of Bft may be a possible strategy for treating patients suffering from IBD.

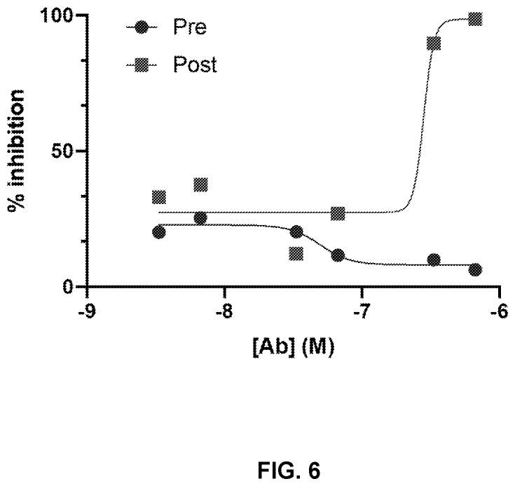

To test this hypothesis, rabbits were immunized with recombinant Bft. Subsequently, serum was obtained and IgG was purified. Purified rabbit IgG from pre- and post-immunization serum was incubated with recombinant Bft1 for 1 hr at 37° C., then added to cultured HT29 cells. Cell culture supernatant was harvested after 18 hours, and the level of E-cadherin release was measured by ELISA. As shown in FIG. 6, rabbit IgG purified from anti-Bft hyper-immune sera but not from pre-immunization sera neutralized Bft activity.

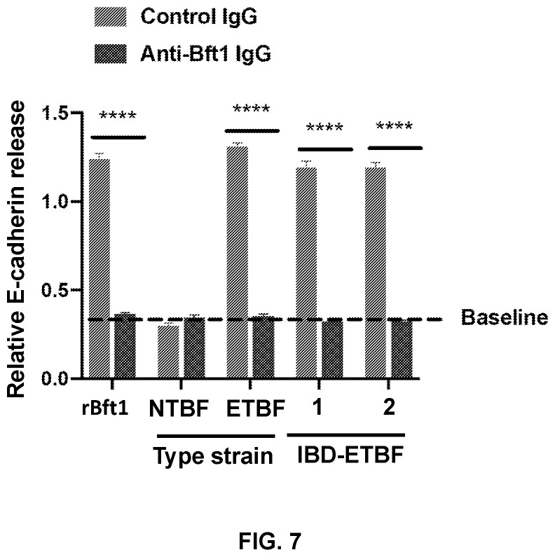

Next, HT29 cellls were treated with the culture supernatant of NTBF or ETBF strains (i.e., containing secreted Bft), with control or anti-Bft rabbit polyclonal antibody for 18 hours. HT29 cell culture supernatant was harvested and E-cadherin release was measured using the standard ELISA. As shown in FIG. 7, anti-bft IgG inhibited the activity of Bft. After treatment with the anti-bft IgG, Bft activity was reduced to baseline levels, indicating a nearly complete inhibition of protease activity.

Taken together, these data demonstrate that an anti-Bft IgG may be used to inhibit the activity of Bft and reduce the pathogenicity of ETBF strains.

### Example 4

### Vaccination of a Patient

To vaccinate a patient against ETBF and thus treat or prevent IBD, a recombinant B. fragilis enterotoxin will be synthesized from a known toxin sequence. The enterotoxin will then be formulated as a vaccine for intramuscular administration. The vaccine composition will be administered to a patient in a therapeutically effective amount, i.e. an amount sufficient to stimulate an immune response against the enterotoxin in the patient.

### Example 5

Treating a Patient Suffering from IBD

In order to treat a patient suffering from an inflammatory bowel disease, a humanized, monoclonal antibody that specifically binds to and neutralizes a B. fragilis enterotoxin will be provided. The antibody will be administered to a patient in a therapeutically effective amount, i.e. an amount sufficient to reduce the amount or the pathogenic effects of an ETBF or an enterotoxin produced by the ETBF. Treatment will be repeated as necessary, until the symptoms if IBD in the patient are absent or reduced to clinically acceptable levels.

### NUMBERED EMBODIMENTS OF THE DISCLOSURE

Notwithstanding the appended claims, the disclosure sets forth the following numbered embodiments:

1. A method of treating a subject diagnosed with Irritable Bowel Disease (IBD), the method comprising administering to the subject an agent to reduce the number or pathogenic effects of a B. fragilis strain, wherein the subject is diagnosed with IBD by detecting the presence of the B. fragilis strain in a biological sample of the patient.

2. The method of embodiment 1, wherein the agent is an antibody.

3. The method of embodiment 2, wherein the antibody is humanized.

4. The method of embodiment 2 or 3, wherein the antibody is monoclonal.

5. The method of any one of embodiments 2-4, wherein the antibody binds to a B. fragilis toxin.

6. The method of embodiment 5, wherein the B. fragilis toxin is BFT1, BFT2, and/or BFT3.

7. The method of embodiment 6, wherein the B. fragilis toxin has a sequence at least 95% or 100% identical to any one of SEQ ID NO: 2-4.

8. The method of embodiment 1, wherein the agent is a vaccine.

9. The method of embodiment 8, wherein the vaccine comprises an inactivated B. fragilis enterotoxin.

10. The method of embodiment 8, wherein the vaccine comprises an inactivated B. fragilis bacterium.

11. The method of embodiment 9, wherein the inactivated B. fragilis enterotoxin is recombinant.

12. The method of any one of embodiments 8-11, wherein the vaccine comprises an adjuvant.

13. The method of embodiment 12, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

14. The method of any one of embodiments 1-13, wherein the IBD is Crohn's disease.

15. The method of embodiment 14, wherein the IBD is ulcerative colitis.

16. The method of any one of embodiments 1-15, wherein the IBD is active IBD.

17. The method of any one of embodiments 1-15, wherein the IBD is inactive IBD.

18. The method of any one of embodiments 1-17, wherein the B. fragilis strain is an Enterotoxigenic strain of B. fragilis.

19. The method of embodiment 18, wherein the B. fragilis strain produces a toxin selected from BFT1, BFT2, and BFT3.

20. The method of embodiment 19, wherein the B. fragilis toxin has a sequence at least 95% or 100% identical to any one of SEQ ID NO: 2-4.

21. The method of any one of embodiments 1-20, wherein the subject is a mammal.

22. The method of embodiment 21, wherein the subject is a human.

23. The method of any one of embodiments 1-22, wherein the subject is at least 18 years of age.

24. The method of any one of embodiments 1-22, wherein the subject is less than 18 years of age.

25. The method of any one of embodiments 1-24, wherein the biological sample is a blood sample.

26. The method of any one of embodiments 1-24, wherein the biological sample is a stool sample.

27. The method of any one of embodiments 1-26, wherein the agent is administered orally.

28. The method of any one of embodiments 1-26, wherein the agent is administered intravenously.

29. The method of any one of embodiments 1-26, wherein the agent is administered intramuscularly.

30. The method of any one of embodiments 1-26, wherein the agent is administered subcutaneously.

31. The method of any one of embodiments 1-30, wherein administering the agent reduces the levels of one or more pro-inflammatory cytokines in the subject.

32. The method of embodiment 31, wherein administering the agent reduces the levels of at least one of TNF and IL-17 in the subject.

33. A method of treating a subject diagnosed with Irritable Bowel Disease (IBD), the method comprising administering to the subject an agent to reduce the expression or activity of a B. fragilis toxin, wherein the subject is diagnosed with IBD by detecting the presence of the B. fragilis toxin in a biological sample of the patient.

34. The method of embodiment 33, wherein the agent is an antibody.

35. The method of embodiment 34, wherein the antibody is humanized.

36. The method of embodiment 34 or 35, wherein the antibody is monoclonal.

37. The method of any one of embodiments 33-36, wherein the antibody binds to the B. fragilis toxin.

38. The method of any one of embodiments 33-37, wherein the B. fragilis toxin is BFT1, BFT2, and/or BFT3.

39. The method of embodiment 38, wherein the B. fragilis toxin has a sequence at least 95% or 100% identical to any one of SEQ ID NO: 2-4.

40. The method of embodiment 30, wherein the agent is a vaccine.

41. The method of embodiment 30, wherein the vaccine comprises an inactivated B. fragilis enterotoxin.

42. The method of embodiment 40, wherein the vaccine comprises an inactivated B. fragilis bacterium.

43. The method of embodiment 41, wherein the inactivated B. fragilis enterotoxin is recombinant.

44. The method of any one of embodiments 40-43, wherein the vaccine comprises an adjuvant.

45. The method of embodiment 44, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

46. The method of any one of embodiments 33-45, wherein the IBD is Crohn's disease.

47. The method of any one of embodiments 33-45, wherein the IBD is ulcerative colitis.

48. The method of any one of embodiments 33-47, wherein the IBD is active IBD.

49. The method of any one of embodiments 33-47, wherein the IBD is inactive IBD.

50. The method of any one of embodiments 33-49, wherein the subject is a mammal.

51. The method of embodiment 50, wherein the subject is a human.

52. The method of any one of embodiments 33-51, wherein the subject is at least 18 years of age.

53. The method of any one of embodiments 33-51, wherein the subject is less than 18 years of age.

54. The method of any one of embodiments 33-53, wherein the biological sample is a blood sample.

55. The method of any one of embodiments 33-53, wherein the biological sample is a stool sample.

56. The method of any one of embodiments 33-55, wherein the agent is administered orally.

57. The method of any one of embodiments 33-55, wherein the agent is administered intravenously.

58. The method of any one of embodiments 33-55, wherein the agent is administered intramuscularly.

59. The method of any one of embodiments 33-55, wherein the agent is administered subcutaneously.

60. The method of any one of embodiments 33-59, wherein administering the agent reduce the levels of one or more pro-inflammatory cytokines in the subject.

61. The method of embodiment 60, wherein administering the vaccine reduces the levels of at least one of TNF and IL-17 in the subject.

62. A method of treating a subject in need thereof, the method comprising detecting the presence of a B. fragilis strain in a biological sample of the subject, and administering to the subject an agent to reduce the number of or pathogenic effects of the B. fragilis strain.

63. A method of treating a subject in need thereof, the method comprising detecting the presence of the B. fragilis toxin in a biological sample of the subject, and administering to the subject an agent to reduce the expression or activity of the B. fragilis toxin.

64. A vaccine composition for treating or preventing an inflammatory bowel disease or disorder, the vaccine composition comprising an inactivated B. fragilis enterotoxin.

65. The vaccine composition of embodiment 64, wherein the inflammatory bowel disease is Crohn's disease or ulcerative colitis.

66. The vaccine composition of any one of embodiments 64-65, wherein the inactivated B. fragilis enterotoxin is recombinant.

67. The vaccine composition of any one of embodiments 64-66, wherein the enterotoxin comprises the amino acid sequence of any one of SEQ ID NO: 2-4.

68. The vaccine composition of any one of embodiments 64-67, wherein the vaccine composition comprises an adjuvant.

69. The vaccine composition of embodiment 68, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, 0M-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

70. The vaccine composition of any one of embodiments 64-69, wherein the vaccine composition is formulated for oral administration or intravenous administration.

71. The vaccine composition of any one of embodiments 64-69, wherein the vaccine composition is formulated for intramuscular administration or subcutaneous administration.

72. A method for treating or preventing an inflammatory bowel disease in a subject, the method comprising administering the vaccine composition of any one of embodiments 64-71 to the subject.

73. The method of embodiment 72, wherein the subject is a mammal.

74. The method of embodiment 73, wherein the subject is a human.

75. The method of any one of embodiments 72-74, wherein the vaccine composition is administered once to the subject.

76. The method of any one of embodiments 72-74, wherein the vaccine composition is administered more than once to the subject.

77. The method of any one of embodiments 72-76, wherein the vaccine composition is administered orally to the subject.

78. The method of any one of embodiments 72-76, wherein the vaccine composition is administered intramuscularly to the subject.

79. The method of any one of embodiments 72-76, wherein the agent is administered intramuscularly to the subject.

80. The method of any one of embodiments 72-76, wherein the agent is administered subcutaneously.

81. The method of any one of embodiments 72-80, wherein administering the vaccine reduces the levels of one or more pro-inflammatory cytokines in the subject.

82. The method of embodiment 81, wherein administering the vaccine reduces the levels of at least one of TNF and IL-17 in the subject.

83. A method of treating or preventing an inflammatory bowel disease in a subject, the method comprising administering to the subject an agent to reduce the number or pathogenic effects of an enterotoxigenic B. fragilis strain.

84. The method of embodiment 83, wherein the agent comprises a vaccine.

85. The method of embodiment 84, wherein the vaccine comprises an inactivated bacterium.

86. The method of embodiment 84, wherein the vaccine comprises an inactivated enterotoxin.

87. The method of any one of embodiments 84-86, wherein the vaccine comprises an adjuvant.

88. The method of embodiment 87, wherein the adjuvant is selected from the group consisting of complete or incomplete Freund's adjuvant, R IBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, 0M-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

89. The method of any one of embodiments 84-88, wherein the vaccine is administered orally to the subject.

90. The method of any one of embodiments 84-88, wherein the vaccine is administered intramuscularly to the subject.

91. The method of any one of embodiments 84-88, wherein the agent is administered intramuscularly.

92. The method of any one of embodiments 84-88, wherein the agent is administered subcutaneously.

93. The method of embodiment 83, wherein the agent comprises an antibody.

94. The method of embodiment 93, wherein the antibody is a monoclonal antibody.

95. The method of any one of embodiments 93-94, wherein the antibody is a humanized antibody.

96. The method of any one of embodiments 93-95, wherein the antibody binds to the enterotoxigenic B. fragilis.

97. The method of any one of embodiments 93-95, wherein the antibody binds to an enterotoxin.

98. The method of embodiment 97, wherein the enterotoxin comprises the amino acid sequence of any one of SEQ ID NO: 2-4.

99. The method of any one of embodiments 93-98, wherein the subject is a mammal.

100. The method of embodiment 99, wherein the subject is a human.

101. The method of any one of embodiments 93-100, wherein administering the agent reduces the levels of one or more pro-inflammatory cytokines in the subject.

102. The method of embodiment 101, wherein administering the agent reduces the levels of at least one of TNF and IL-17 in the subject.

103. A method of treating or preventing an inflammatory bowel disease in a subject, the method comprising administering to the subject an agent to reduce the effects of an enterotoxin produced by a B. fragilis strain.

104. The method of embodiment 103, wherein the agent comprises a vaccine.

105. The method of embodiment 104, wherein the vaccine comprises an inactivated bacterium.

106. The method of embodiment 104, wherein the vaccine comprises an inactivated enterotoxin.

107. The method of any one of embodiments 104-106, wherein the vaccine comprises an adjuvant.

108. The method of embodiment 107, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, 0M-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

109. The method of any one of embodiments 104-108, wherein the vaccine is administered orally or intravenously to the subject.

110. The method of any one of embodiments 104-108, wherein the vaccine is administered intramuscularly or subcutaneously to the subject.

111. The method of embodiment 98, wherein the agent comprises an antibody.

112. The method of embodiment 111, wherein the antibody is a monoclonal antibody.

113. The method of any one of embodiments 111-112, wherein the antibody is a humanized antibody.

114. The method of any one of embodiments 111-113, wherein the antibody binds to the enterotoxigenic B. fragilis.

115. The method of any one of embodiments 111-113, wherein the antibody binds to an enterotoxin.

116. The method of embodiment 115, wherein the enterotoxin comprises the amino acid sequence of any one of SEQ ID NO: 2-4.

117. The method of any one of embodiments 103-116, wherein the subject is a mammal.

118. The method of embodiment 117, wherein the subject is a human.

119. The method of any one of embodiments 103-118, wherein administering the agent reduces the levels of one or more pro-inflammatory cytokines in the subject.

120. The method of embodiment 119, wherein administering the vaccine reduces the levels of at least one of TNF and IL-17 in the subject.

121. A method of treating or preventing an inflammatory bowel disease in a subject, the method comprising administering to the subject an agent to reduce the number or pathogenic effects of a B. fragilis strain.

122. The method of embodiment 121, wherein the agent comprises a vaccine.

123. The method of embodiment 122, wherein the vaccine comprises an inactivated bacterium.

124. The method of embodiment 122, wherein the vaccine comprises an inactivated enterotoxin.

125. The method of any one of embodiments 122-124, wherein the vaccine comprises an adjuvant.

126. The method of embodiment 125, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

127. The method of any one of embodiments 122-126, wherein the vaccine is administered orally or intravenously to the subject.

128. The method of any one of embodiments 122-126, wherein the vaccine is administered intramuscularly or subcutaneously to the subject.

129. The method of embodiment 121, wherein the agent comprises an antibody.

130. The method of embodiment 129, wherein the antibody is a monoclonal antibody.

131. The method of any one of embodiments 129-130, wherein the antibody is a humanized antibody.

132. The method of any one of embodiments 129-131, wherein the antibody binds to the enterotoxigenic B. fragilis.

133. The method of any one of embodiments 129-131, wherein the antibody binds to an enterotoxin.

134. The method of embodiment 133, wherein the enterotoxin comprises the amino acid sequence of any one of SEQ ID NO: 2-4.

135. The method of any one of embodiments 121-134, wherein the subject is a mammal.

136. The method of embodiment 135, wherein the subject is a human.

The disclosures of each and every patent, patent application, and publication cited herein are hereby incorporated herein by reference in their entirety. While this disclosure contains references to specific embodiments, it is apparent that other embodiments and variations of this disclosure may be devised by others skilled in the art without departing from the true spirit and scope of the invention. The appended claims are intended to be construed to include all such embodiments and equivalent variations.

What is claimed is:

1. A method of treating a subject diagnosed with Irritable Bowel Disease (IBD), the method comprising administering to the subject an agent to reduce the number or pathogenic effects of a B. fragilis strain,
wherein the subject is diagnosed with IBD by detecting the presence of the B. fragilis strain in a biological sample of the patient.

2. The method of claim 1, wherein the agent is an antibody.

3. The method of claim 2, wherein the antibody is humanized.

4. The method of claim 2 or 3, wherein the antibody is monoclonal.

5. The method of any one of claims 2-4, wherein the antibody binds to a B. fragilis toxin.

6. The method of claim 5, wherein the B. fragilis toxin is BFT1, BFT2, and/or BFT3.

7. The method of claim 6, wherein the B. fragilis toxin has a sequence at least 95% or 100% identical to any one of SEQ ID NO: 2-4.

8. The method of claim 1, wherein the agent is a vaccine.

9. The method of claim 8, wherein the vaccine comprises an inactivated B. fragilis enterotoxin.

10. The method of claim 8, wherein the vaccine comprises an inactivated B. fragilis bacterium.

11. The method of claim 9, wherein the inactivated B. fragilis enterotoxin is recombinant.

12. The method of any one of claims 8-11, wherein the vaccine comprises an adjuvant.

13. The method of claim 12, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

14. The method of any one of claims 1-13, wherein the IBD is Crohn's disease.

15. The method of claim 14, wherein the IBD is ulcerative colitis.

16. The method of any one of claims 1-15, wherein the IBD is active IBD.

17. The method of any one of claims 1-15, wherein the IBD is inactive IBD.

18. The method of any one of claims 1-17, wherein the B. fragilis strain is an Enterotoxigenic strain of B. fragilis.

19. The method of claim 18, wherein the B. fragilis strain produces a toxin selected from BFT1, BFT2, and BFT3.

20. The method of claim 19, wherein the B. fragilis toxin has a sequence at least 95% or 100% identical to any one of SEQ ID NO: 2-4.

21. The method of any one of claims 1-20, wherein the subject is a mammal.

22. The method of claim 21, wherein the subject is a human.

23. The method of any one of claims 1-22, wherein the subject is at least 18 years of age.

24. The method of any one of claims 1-22, wherein the subject is less than 18 years of age.

25. The method of any one of claims 1-24, wherein the biological sample is a blood sample.

26. The method of any one of claims 1-24, wherein the biological sample is a stool sample.

27. The method of any one of claims 1-26, wherein the agent is administered orally.

28. The method of any one of claims 1-26, wherein the agent is administered intravenously.

29. The method of any one of claims 1-26, wherein the agent is administered intramuscularly.

30. The method of any one of claims 1-26, wherein the agent is administered subcutaneously.

31. The method of any one of claims 1-30, wherein administering the agent reduces the levels of one or more pro-inflammatory cytokines in the subject.

32. The method of claim 31, wherein administering the agent reduces the levels of at least one of TNF and IL-17 in the subject.

33. A method of treating a subject diagnosed with Irritable Bowel Disease (IBD), the method comprising administering to the subject an agent to reduce the expression or activity of a B. fragilis toxin, wherein the subject is diagnosed with IBD by detecting the presence of the B. fragilis toxin in a biological sample of the patient.

34. The method of claim 33, wherein the agent is an antibody.

35. The method of claim 34, wherein the antibody is humanized.

36. The method of claim 34 or 35, wherein the antibody is monoclonal.

37. The method of any one of claims 33-36, wherein the antibody binds to the B. fragilis toxin.

38. The method of any one of claims 33-37, wherein the B. fragilis toxin is BFT1, BFT2, and/or BFT3.

39. The method of claim 38, wherein the B. fragilis toxin has a sequence at least 95% or 100% identical to any one of SEQ ID NO: 2-4.

40. The method of claim 30, wherein the agent is a vaccine.

41. The method of claim 30, wherein the vaccine comprises an inactivated B. fragilis enterotoxin.

42. The method of claim 40, wherein the vaccine comprises an inactivated B. fragilis bacterium.

43. The method of claim 41, wherein the inactivated B. fragilis enterotoxin is recombinant.

44. The method of any one of claims 40-43, wherein the vaccine comprises an adjuvant.

45. The method of claim 44, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

46. The method of any one of claims 33-45, wherein the IBD is Crohn's disease.

47. The method of any one of claims 33-45, wherein the IBD is ulcerative colitis.

48. The method of any one of claims 33-47, wherein the IBD is active IBD.

49. The method of any one of claims 33-47, wherein the IBD is inactive IBD.

50. The method of any one of claims 33-49, wherein the subject is a mammal.

51. The method of claim 50, wherein the subject is a human.

52. The method of any one of claims 33-51, wherein the subject is at least 18 years of age.

53. The method of any one of claims 33-51, wherein the subject is less than 18 years of age.

54. The method of any one of claims 33-53, wherein the biological sample is a blood sample.

55. The method of any one of claims 33-53, wherein the biological sample is a stool sample.

56. The method of any one of claims 33-55, wherein the agent is administered orally.

57. The method of any one of claims 33-55, wherein the agent is administered intravenously.

58. The method of any one of claims 33-55, wherein the agent is administered intramuscularly.

59. The method of any one of claims 33-55, wherein the agent is administered subcutaneously.

60. The method of any one of claims 33-59, wherein administering the agent reduce the levels of one or more pro-inflammatory cytokines in the subject.

61. The method of claim 60, wherein administering the vaccine reduces the levels of at least one of TNF and IL-17 in the subject.

62. A method of treating a subject in need thereof, the method comprising detecting the presence of a B. fragilis strain in a biological sample of the subject, and administering to the subject an agent to reduce the number of or pathogenic effects of the B. fragilis strain.

63. A method of treating a subject in need thereof, the method comprising detecting the presence of the B. fragilis toxin in a biological sample of the subject, and
administering to the subject an agent to reduce the expression or activity of the B. fragilis toxin.

64. A vaccine composition for treating or preventing an inflammatory bowel disease or disorder, the vaccine composition comprising an inactivated B. fragilis enterotoxin.

65. The vaccine composition of claim 64, wherein the inflammatory bowel disease is Crohn's disease or ulcerative colitis.

66. The vaccine composition of any one of claims 64-65, wherein the inactivated B. fragilis enterotoxin is recombinant.

67. The vaccine composition of any one of claims 64-66, wherein the enterotoxin comprises the amino acid sequence of any one of SEQ ID NO: 2-4.

68. The vaccine composition of any one of claims 64-67, wherein the vaccine composition comprises an adjuvant.

69. The vaccine composition of claim 68, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

70. The vaccine composition of any one of claims 64-69, wherein the vaccine composition is formulated for oral administration or intravenous administration.

71. The vaccine composition of any one of claims 64-69, wherein the vaccine composition is formulated for intramuscular administration or subcutaneous administration.

72. A method for treating or preventing an inflammatory bowel disease in a subject, the method comprising administering the vaccine composition of any one of claims 64-71 to the subject.

73. The method of claim 72, wherein the subject is a mammal.

74. The method of claim 73, wherein the subject is a human.

75. The method of any one of claims 72-74, wherein the vaccine composition is administered once to the subject.

76. The method of any one of claims 72-74, wherein the vaccine composition is administered more than once to the subject.

77. The method of any one of claims 72-76, wherein the vaccine composition is administered orally to the subject.

78. The method of any one of claims 72-76, wherein the vaccine composition is administered intramuscularly to the subject.

79. The method of any one of claims 72-76, wherein the agent is administered intramuscularly to the subject.

80. The method of any one of claims 72-76, wherein the agent is administered subcutaneously.

81. The method of any one of claims 72-80, wherein administering the vaccine reduces the levels of one or more pro-inflammatory cytokines in the subject.

82. The method of claim 81, wherein administering the vaccine reduces the levels of at least one of TNF and IL-17 in the subject.

83. A method of treating or preventing an inflammatory bowel disease in a subject, the method comprising administering to the subject an agent to reduce the number or pathogenic effects of an enterotoxigenic B. fragilis strain.

84. The method of claim 83, wherein the agent comprises a vaccine.

85. The method of claim 84, wherein the vaccine comprises an inactivated bacterium.

86. The method of claim 84, wherein the vaccine comprises an inactivated enterotoxin.

87. The method of any one of claims 84-86, wherein the vaccine comprises an adjuvant.

88. The method of claim 87, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

89. The method of any one of claims 84-88, wherein the vaccine is administered orally to the subject.

90. The method of any one of claims 84-88, wherein the vaccine is administered intramuscularly to the subject.

91. The method of any one of claims 84-88, wherein the agent is administered intramuscularly.

92. The method of any one of claims 84-88, wherein the agent is administered subcutaneously.

93. The method of claim 83, wherein the agent comprises an antibody.

94. The method of claim 93, wherein the antibody is a monoclonal antibody.

95. The method of any one of claims 93-94, wherein the antibody is a humanized antibody.

96. The method of any one of claims 93-95, wherein the antibody binds to the enterotoxigenic B. fragilis.

97. The method of any one of claims 93-95, wherein the antibody binds to an enterotoxin.

98. The method of claim 97, wherein the enterotoxin comprises the amino acid sequence of any one of SEQ ID NO: 2-4.

99. The method of any one of claims 93-98, wherein the subject is a mammal.

100. The method of claim 99, wherein the subject is a human.

101. The method of any one of claims 93-100, wherein administering the agent reduces the levels of one or more pro-inflammatory cytokines in the subject.

102. The method of claim 101, wherein administering the agent reduces the levels of at least one of TNF and IL-17 in the subject.

103. A method of treating or preventing an inflammatory bowel disease in a subject, the method comprising administering to the subject an agent to reduce the effects of an enterotoxin produced by a B. fragilis strain.

104. The method of claim 103, wherein the agent comprises a vaccine.

105. The method of claim 104, wherein the vaccine comprises an inactivated bacterium.

106. The method of claim 104, wherein the vaccine comprises an inactivated enterotoxin.

107. The method of any one of claims 104-106, wherein the vaccine comprises an adjuvant.

108. The method of claim 107, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

109. The method of any one of claims 104-108, wherein the vaccine is administered orally or intravenously to the subject.

110. The method of any one of claims 104-108, wherein the vaccine is administered intramuscularly or subcutaneously to the subject.

111. The method of claim 98, wherein the agent comprises an antibody.

112. The method of claim 111, wherein the antibody is a monoclonal antibody.

113. The method of any one of claims 111-112, wherein the antibody is a humanized antibody.

114. The method of any one of claims 111-113, wherein the antibody binds to the enterotoxigenic B. fragilis.

115. The method of any one of claims 111-113, wherein the antibody binds to an enterotoxin.

116. The method of claim 115, wherein the enterotoxin comprises the amino acid sequence of any one of SEQ ID NO: 2-4.

117. The method of any one of claims 103-116, wherein the subject is a mammal.

118. The method of claim 117, wherein the subject is a human.

119. The method of any one of claims 103-118, wherein administering the agent reduces the levels of one or more pro-inflammatory cytokines in the subject.

120. The method of claim 119, wherein administering the vaccine reduces the levels of at least one of TNF and IL-17 in the subject.

121. A method of treating or preventing an inflammatory bowel disease in a subject, the method comprising administering to the subject an agent to reduce the number or pathogenic effects of a B. fragilis strain.

122. The method of claim 121, wherein the agent comprises a vaccine.

123. The method of claim 122, wherein the vaccine comprises an inactivated bacterium.

124. The method of claim 122, wherein the vaccine comprises an inactivated enterotoxin.

125. The method of any one of claims 122-124, wherein the vaccine comprises an adjuvant.

126. The method of claim 125, wherein the adjuvant is selected from the group consisting of complete or incomplete Freund's adjuvant, RIBI, KLH peptide, cholera toxin, E. coli heat-labile toxin, E. coli enterotoxin, salmonella toxin, nanoparticle-based adjuvant, aluminum salts, calcium phosphate, liposomes, virosomes, cochleates, eurocine, archaeal lipids, ISCOMS, microparticles, monophosphoryl lipid (MPL), N-acetyl-muramyl-L-alanyl-D-isoglutamine (MDP), Detox, AS04, AS02, AS01, OM-174, OM-triacyl, oligonucleotides, double-stranded RNA, pathogen-associated molecular patterns (PAMPs), TLR ligands, saponins, chitosan, a-galactosylceramide, small-molecule immune potentiators (SMIPs), a cytokine, a chemokine, DC Choi, PLA (polylactic acid) microparticles, PLG (poly[lactide-co-glycolide]) microparticles, Poly(DL-lactide-co-glycolide) microparticles, polystyrene (latex) microparticles, proteosomes, and 3′,5′-Cyclic diguanylic acid (c-di-GMP).

127. The method of any one of claims 122-126, wherein the vaccine is administered orally or intravenously to the subject.

128. The method of any one of claims 122-126, wherein the vaccine is administered intramuscularly or subcutaneously to the subject.

129. The method of claim 121, wherein the agent comprises an antibody.

130. The method of claim 129, wherein the antibody is a monoclonal antibody.

131. The method of any one of claims 129-130, wherein the antibody is a humanized antibody.

132. The method of any one of claims 129-131, wherein the antibody binds to the enterotoxigenic B. fragilis.

133. The method of any one of claims 129-131, wherein the antibody binds to an enterotoxin.

134. The method of claim 133, wherein the enterotoxin comprises the amino acid sequence of any one of SEQ ID NO: 2-4.

135. The method of any one of claims 121-134, wherein the subject is a mammal.

136. The method of claim 135, wherein the subject is a human.
